(19) United States
(12) Patent Application Publication
(10) Pub. No.: US 2009/0203954 A1
(43) Pub. Date: Aug. 13, 2009

(54) Magnetic-Field Applicator

(76) Inventors: Dietrich H. W. Grönemeyer, Sprockhövel (DE); Martin Busch, Witten (DE); Frank Rüdiger, Berlin (DE); Julia Busch, Witten (DE)

Correspondence Address: COLLARD & ROE, P.C., 1077 NORTHERN BOULEVARD, ROSLYN, NY, 11576, US

(21) Appl. No.: 12/087,823

(22) PCT Filed: Dec. 2, 2006
(86) PCT No.: PCT/EP2006/011588

§ 371 Date: Feb. 3, 2009

(30) Foreign Application Priority Data
Dec. 20, 2005 (DE) 10 2005 060 834.5

(51) Int. Cl. A61N 2/04

(52) U.S. Cl. 600/13

(57) ABSTRACT

The invention relates to a magnetic-field applicator for heating magnetic or magnetizable substances or particles in human or animal tissue having at least two magnet coils (1) for producing a magnetic alternating field, wherein the arrangement of the magnet coils (1) is matched to the body part (2) to be treated in such a way that the magnet coils (1) are on both sides of the body part (2) and the magnetic field produced is concentrated on the body part (2) to be treated. In this way, the volume within which the magnetic field needs to be produced is considerably reduced in comparison with whole-body applicators, which is associated with a lower performance of the magnetic-field applicator for producing the desired magnetic field strength. Furthermore, the magnetic alternating field can be focused on specific areas of the body part (2) to be treated.

The invention relates to a magnetic-field applicator for heating magnetic or magnetizable substances or particles in human or animal tissue, having at least two magnetic coils for generating a magnetic alternating field.

Treatment of cancer can take place in different ways, whereby surgical removal of a tumor, chemotherapy, and radiation therapy should be particularly mentioned. However, all these treatment methods have various disadvantages. For example, operative removal of a tumor in the case of cancer in an advanced stage, after the formation of metastases, or depending on the location of the tumor, is possible only with difficulty. Chemotherapy, on the other hand, which is frequently used together with the other methods mentioned, has a systemic effect on the entire body and is connected with significant side effects.

Another, more recent method for cancer therapy is so-called hyperthermia or thermal ablation, in which tumor tissue is heated to temperatures above 41° C. One speaks of hyperthermia in the temperature range between 41° and 46° C., in which controlled decomposition of the tumor occurs, supported by the body. At higher temperatures, above approximately 47° C., acute destruction of the cells as the result of the high temperature occurs. Such a process is called thermal ablation.

In the case of hyperthermia or thermal ablation, it is known from DE 199 37 493 C2, for example, to heat magnetic or magnetizable substances in the tissue by means of applying a magnetic alternating field. For this purpose, magnetizable substances are first introduced into the region of the tumor, whereby these can be magnetic liquids, for example. In the case of other methods known from the state of the art, for example according to U.S. Pat. No. 5,197,940, thermoseeds, which consist of ferromagnetic materials, are introduced into the tumor region. However, this introduction of the thermoseeds takes place surgically, in relatively complicated manner.

WO 97/43005 A1 proposes allowing magnetizable microcapsules to get into the tumor region through the bloodstream. EP 1 001 811 B1 describes another advantageous method, according to which iron nanoparticles are provided with two sheaths, whereby the inner sheath has positively charged functional groups, which allow easy introduction of the nanoparticles into the interior of the tumor cells, while the outer sheath compensates the positive charges, so that the particles as a whole appear neutral or negative towards the outside. The latter is important in order to achieve good distribution of the particles in the tissue. In this manner, paramagnetic particles accumulate strongly in the tumor tissue, so that by applying a magnetic alternating field, the tumor tissue is specifically heated up, while adjacent, healthy tissue remains unaffected, to a great extent.

Furthermore, hyperthermia treatment can also be combined with chemotherapy, in that some chemotherapeutic agents only develop their full effectiveness at an elevated temperature. If the elevated temperature is generated only in the tumor tissue, more effective chemotherapy, subject to fewer side effects, is also achieved in this way.

In the case of the treatments known until now, a whole-body magnetic-field applicator is used. In this connection, it has proven to be disadvantageous that very great power is needed to build up a magnetic field of the corresponding frequency and amplitude.

Proceeding from this state of the art, the task is therefore posed of making available a magnetic-field applicator that can be used to carry out treatment of tumor tissue just as effectively as according to the state of the art, but on the other hand, the power that is required to generate the magnetic field is clearly reduced.

This task is accomplished, according to the invention, by means of a magnetic-field applicator for heating magnetic or magnetizable substances or particles in human or animal tissue, having at least two magnetic coils for generating a magnetic alternating field, whereby the arrangement of the magnetic coils is coordinated with the body part to be treated, in such a manner that the magnetic coils lie on both sides of the body part, and the magnetic field that is generated is concentrated on the body part to be treated.

The invention is based on the idea that the power can be significantly reduced if the volume in which the magnetizable substances, for example magnetite particles, are heated by means of the magnetic alternating field, is reduced. This is possible for many areas of tumor therapy in the human body, particularly in the case of treatment of the extremities and of the female breast. Accordingly, the invention particularly relates to a magnetic-field applicator in which the arrangement of the magnetic coils is coordinated with the treatment of a female breast, so that the magnetic field that is generated focuses on the latter. Such a magnetic-field applicator has particular significance in medicine, since breast cancer is very widespread. Fundamentally, however, the invention can also be used for treatment of other body parts, with a slightly different structure.

The magnetic coils can be disposed parallel to one another, whereby the body part to be treated is introduced between the magnetic coils. In the case of magnetic coils disposed parallel to one another, the magnetic-field lines run parallel to one another, so that the magnetic field that prevails at a specific location is relatively easy to adjust.

Alternatively to this, however, it is also possible to dispose the magnetic coils at a certain angle, so that the surfaces of the magnetic coils run parallel to the surfaces of the body part to be treated. In this connection, the sides from which the magnetic-field lines exit in the direction of the body part to be treated are understood to be the surfaces of the magnetic coils. The arrangement of the magnetic coils at an angle has the advantage that in this manner, the magnetic-field applicator can be better adapted to the body part to be treated, particularly if this is a female breast.

In order to achieve further optimization of the magnetic-field applicator, the magnetic coils can be configured so that they can be displaced relative to one another. In this manner, the breast can be introduced into the magnetic-field applicator, for example, and then the magnetic coils can be moved towards one another, and the breast can thus be fixed in place. Such a magnetic-field applicator, having displaceable magnetic coils, makes the introduction of the body part to be treated into the magnetic-field applicator simpler, on the one hand, and on the other hand brings about improved adaptability to the corresponding body parts of different patients.

A further improvement in the adjustability of the magnetic-field applicator can be achieved in that the magnetic coils are mounted so as to rotate. In this manner, the magnetic field can be focused on specific positions. The treatment of a tumor thus becomes clearly more specific, since the magnetic field can be focused precisely on the location of the tumor, if necessary, bringing about more effective treatment and furthermore avoiding harm to the healthy tissue surrounding the tumor. It is particularly advantageous to configure the magnetic coils so that they can be both displaced and rotated. In addition, the rotating configuration of the magnetic coils also takes the special anatomical needs of the individual patient into account.

It is advantageous if the magnetic-field applicator comprises at least four, particularly preferably at least six magnetic coils, so that at least two or three magnetic coils, respectively, are situated on one side of the body part to be treated, in each instance. Ideally, each individual magnetic coil can be rotated and/or displaced separately, in order to be able, in this manner, to focus the magnetic field on the center or an edge region of the body part to be treated, depending on the location of the tumor.

The frequency of the generated magnetic alternating field typically lies in a range of 10 to 500 kHz. For excitation of magnetite particles introduced into the tumor, a magnetic alternating field of 50 to 150 kHz is particularly preferred, 80 to 120 kHz is very particularly preferred, i.e. a range around approximately 100 kHz.

Of course, the magnetic field does not have to be built up by pure magnetic coils, but rather, the magnetic coils can have pole cores, pole shoes, and an iron pole plate in the form of a yoke, as is fundamentally usual in the case of electromagnets in the state of the art. In particular, the pole cores, the magnetic yoke and/or the pole shoes can consist of ferrites, whereby these are composite ferrite modules. In this connection, there can be gaps between the individual ferrite plates, which serve for cooling.

The invention will be explained in greater detail using the attached figures.

These show:

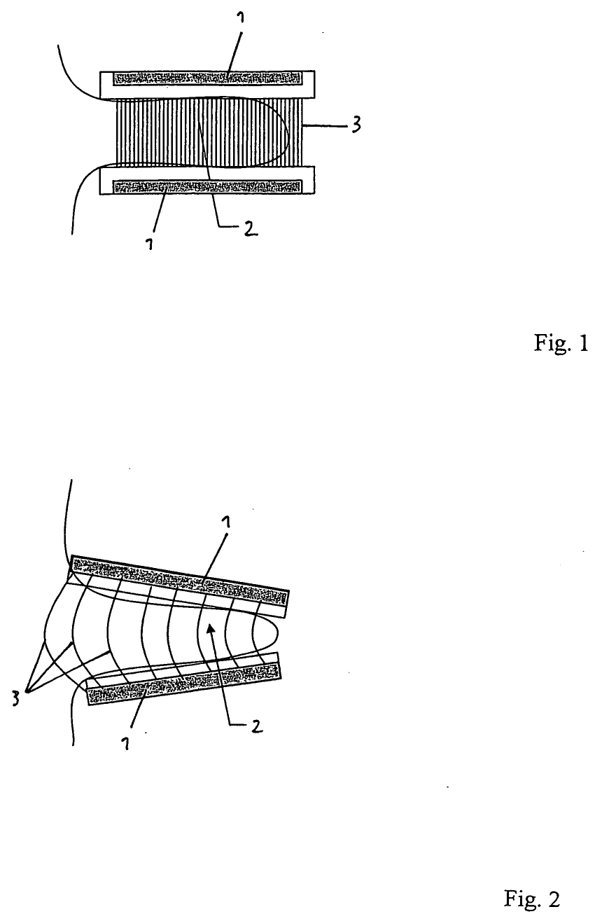

FIG. 1: A magnetic-field applicator according to the invention, in accordance with a first embodiment;

FIG. 2: a magnetic-field applicator according to the invention, in accordance with a second embodiment;

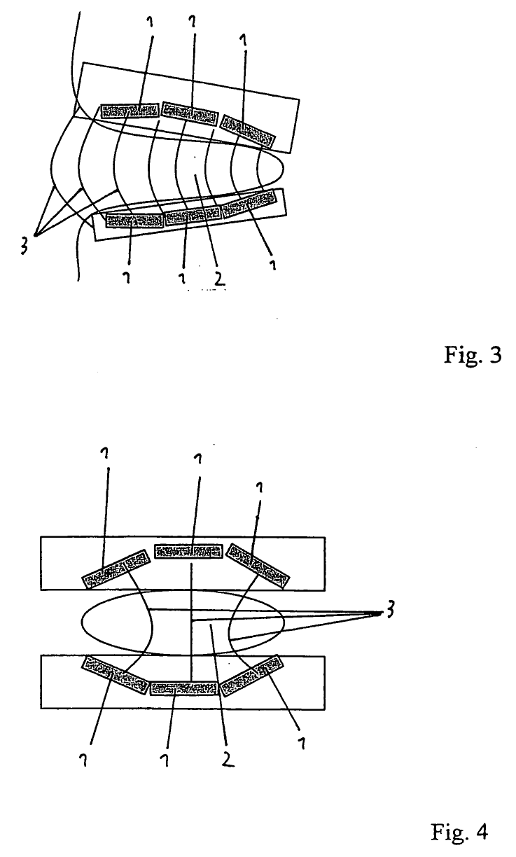

FIG. 3: a magnetic-field applicator according to the invention, in accordance with a third embodiment;

FIG. 4: the magnetic-field applicator from FIG. 3 in a different setting.

FIG. 1 shows a magnetic-field applicator in which the magnetic coils 1 are disposed parallel to one another. The body part 2 to be treated, here a female breast, is brought between the surfaces of the magnetic coils 1, i.e. components of the electromagnet that follow them, whereby in this case, the magnetic-field lines 3 run parallel to one another, and uniformly cover the body part 2 to be treated.

FIG. 2 shows a similar embodiment of the magnetic-field applicator, but it differs from the one shown in FIG. 1 in that here, the magnetic coils 1 are disposed at an angle relative to one another, so that the gap in the field application between the magnetic coils 1 can be better coordinated with the body part 2 to be treated. In this case, however, the magnetic-field lines 3 do not run uniformly parallel, and for this reason, the magnetic field that acts on different regions of the body part 2 to be treated varies in intensity.

FIG. 3 shows an embodiment of the magnetic-field applicator according to the invention in which the magnetic coils 1 are configured so that they can rotate. This has the particular advantage that the magnetic field can be precisely coordinated with the tumor to be treated. For example, the precise location of the tumor can be determined with previously taken image data sets from known imaging methods such as CT and MR, in order to subsequently generate a magnetic field that is optimally coordinated with this tumor. The progression of the magnetic-field lines 3 through the body part 2 to be treated therefore also varies, depending on the setting of the magnetic coils 1.

Finally, in FIG. 4, a different setting of the magnetic coil 1 in the magnetic-field applicator from FIG. 3 is shown, whereby here, the magnetic coils 1 were set in such a manner that the magnetic field is focused on the center of the body part 2 to be treated. Of course, the magnetic field can also be moved farther to the left or the right, by means of corresponding rotation and displacement of the magnetic coils 1. In addition, it should be noted that the magnetic coils 1 can have not only the ability to rotate about an axis perpendicular to the plane of the paper, shown here, but also the ability to rotate about other axes, for example parallel to the plane of the paper, in order to thereby be able to focus the magnetic field in three dimensions, within the tissue to be treated.

1. Magnetic-field applicator for heating magnetic or magnetizable substances or particles in human or animal tissue, having at least two magnetic coils (1) for generating a magnetic alternating field,
wherein
the arrangement of the magnetic coils (1) is coordinated with the body part (2) to be treated, in such a manner that the magnetic coils (1) lie on both sides of the body part (2), and the magnetic field that is generated is concentrated on the body part (2) to be treated.

2. Magnetic-field applicator according to claim 1, wherein the body part (2) to be treated is a female breast.

3. Magnetic-field applicator according to claim 1, wherein the magnetic coils (1) are disposed parallel to one another.

4. Magnetic-field applicator according to claim 1, wherein the magnetic coils (1) form an angle relative to one another, so that the surfaces of the magnetic coils (1) run parallel to the surfaces of the body part (2) to be treated.

5. Magnetic-field applicator according to claim 1, wherein the magnetic coils (1) are displaceable relative to one another.

6. Magnetic-field applicator according to claim 1, wherein the magnetic coils (1) are mounted so as to rotate.

7. Magnetic-field applicator according to claim 5, wherein the magnetic field can be focused on specific parts of the body part (2) to be treated, by means of displacement and/or rotation of the magnetic coils (1).

8. Magnetic-field applicator according to claim 1, wherein the magnetic-field applicator comprises at least four magnetic coils (1).

9. Magnetic-field applicator according to claim 8, wherein the magnetic-field applicator comprises at least six magnetic coils (1).

10. Magnetic-field applicator according to claim 1, wherein the frequency of the magnetic alternating field amounts to 10 to 500 kHz.

11. Magnetic-field applicator according to claim 10, wherein the frequency of the magnetic alternating field amounts to 50 to 150 kHz, preferably 80 to 120 kHz.

12. Magnetic-field applicator according to claim 1, wherein the magnetic coils (1) have pole cores made of ferrites.
